(19) United States
(12) Patent Application Publication
(10) Pub. No.: US 2026/0191476 A1
(43) Pub. Date: Jul. 9, 2026

(54) METHOD OF PREPROCESSING BIO-COMPONENT MEASUREMENT DATA FOR GROWTH PREDICTION

(71) Applicant: GP CO., LTD., Gwangmyeong-si, Gyeonggi-do (KR)

(72) Inventors: Je Hyeok Seong, Gwangmyeong-si, Gyeonggi-do (KR); Ji Hun Kim, Gwacheon-si, Gyeonggi-do (KR); Do Hyun Chun, Seoul (KR); Jong Ho Kang, Seoul (KR)

(73) Assignee: GP CO., LTD., Gwangmyeong-si, Gyeonggi-do (KR)

(21) Appl. No.: 18/863,865

(22) PCT Filed: Aug. 30, 2024
(86) PCT No.: PCT/KR2024/013006

§ 371 Date: Nov. 7, 2024

(30) Foreign Application Priority Data
Aug. 30, 2023 (KR) 10-2023-0114662

(51) Int. Cl. A61B 5/00

(52) U.S. Cl. CPC A61B 5/7275; A61B 5/4869; A61B 2503/06

(57) ABSTRACT

The present disclosure relates to a method, apparatus, and computer program for preprocessing input data for predicting growth of children or adolescents. A method of preprocessing bio-component measurement data for growth prediction according to an exemplary embodiment of the present disclosure may include receiving physical data of a subject, generating a first variable based on the physical data of the subject, and determining an error in the physical data by comparing the first variable with a preset value.

### TECHNICAL FIELD

The present disclosure relates to a method, apparatus, and computer program for preprocessing input data for predicting growth of children or adolescents.

### BACKGROUND ART

With the recent development of artificial intelligence technologies, the artificial intelligence technologies are being applied to various fields. Instead of existing data processing methods, methods of generating additional information by extracting features inherent in data through neural network models have been developed and used.

Recently, the artificial intelligence technology has gone beyond simply tracking and detecting objects and is also being applied to train a past history and derive current features that reflect future predictions or time series change information.

In addition, in artificial intelligence that trains data, the quality of data is known to be one of the key factors in the performance of artificial intelligence. For example, leading companies with the most advanced autonomous driving technology based on the artificial intelligence are emphasizing that securing high-quality data is key.

Among these, the predictive analysis is a technology in areas Of statistics and data mining that extracts information from data and uses the extracted information to predict trends and behavior patterns. This predictive analysis may be applied to all areas where decisions are needed based on information obtained from data. The core of predictive analysis is understanding the relationships between variables and then predicting unknown variables.

For this purpose, various approaches are being used depending on the data characteristics and prediction target.

Among various fields that require the predictive analysis, there is the field of physical growth in children and adolescents. There is a lot of interest among parents and adolescents about when height growth will occur and how much growth will occur.

Regarding the conventional prediction of height growth, a method of predicting a growth plate by taking an X-ray or analyzing a relationship with genetic/environmental factors has been proposed (Korean Patent No. 10-2075743, Korean Patent No. 10-1866208), and a method of making physical data of sample subjects having different measurement times or number of measurements into a form suitable for training a growth prediction model has been proposed (Korean Patent No. 10-2198302).

Since the children and adolescents have growth stages with different features, it is possible to increase the reliability of providing solutions through predicted data and analysis by considering the growth states.

In particular, since the children and adolescents include a period of rapid physical change according to the growth stage, it is necessary to first determine the errors in the input (or measurement) data (bio-component data) that can secure the high-quality data to increase the accuracy of growth prediction and the performance of the artificial intelligence learning.

### Disclosure

### Technical Problem

One of the various tasks of the present disclosure provides a method, apparatus, and computer program for preprocessing input data for predicting growth of children and adolescents, providing customized solutions for each growth stage, etc.

### Technical Solution

According to an exemplary embodiment of the present disclosure, a method of preprocessing bio-component measurement data for growth prediction performed by a computing device includes receiving physical data of a subject, generating a first variable based on the physical data of the subject, and determining an error in the physical data by comparing the first variable with a preset value.

The receiving of the physical data of the subject may include receiving bio-component data of the subject, and receiving identification data of the subject.

The method may further include, after receiving the physical data of the subject, generating first data by connecting the bio-component data of the subject and the identification data of the subject.

The method may further include, after generating the first data, deleting the bio-component data when a preset value is measured in the bio-component data.

The first variable may be generated based on the bio-component data of the subject.

The first variable may be generated by a sum of fat free mass, soft lean mass, and osseous mineral among the bio-component data of the subject.

The determining of the error may include comparing the first variable with a value of any one of the bio-component data.

The determining of the error may further include determining whether a monthly age of the subject among the identification data corresponds to a preset criterion before comparing the first variable with a value of any one of the bio-component data.

The method may further include, when a result value of comparing the first variable with any one value of the bio-component data corresponds to a preset range, deleting the bio-component data.

The method may further include, when a result value of comparing the first variable with any one value of the bio-component data does not correspond to a preset range, generating second data.

According to an exemplary embodiment of the present disclosure, there may be provided a program stored in a computer-readable recording medium including a program code for executing the method of preprocessing height measurement data described above.

According to an exemplary embodiment of the present disclosure, there may be provided a computer-readable recording medium on which a program for executing the method of preprocessing bio-component measurement data for growth prediction described above.

According to another exemplary embodiment of the present disclosure, an apparatus for preprocessing bio-component measurement data for growth prediction includes an input unit that receives physical data of a subject, a variable generation unit that generates a first variable based on the physical data of the subject, and an error determination unit that determines an error in the physical data by comparing the first variable with a preset value.

The input unit may include a first input unit that receives bio-component data of the subject, and a second input unit that receives identification data of the subject.

The apparatus may further include a connection unit that connects data received through the first input unit and the second input unit.

Each feature of the above-described embodiments may be implemented in combination in other embodiments unless inconsistent with or exclusive of the other embodiments.

### Advantageous Effects

According to various embodiments Of the present disclosure, when predicting the growth of children and adolescents, by determining the errors in bio-component data measured for predicting the growth of children and adolescents, it is possible to increase the accuracy of growth prediction.

According to various embodiments of the present disclosure, when providing solution necessary for the growth of children and adolescents, by eliminating errors in input data, it is possible to increase the accuracy of growth prediction.

According to various embodiments of the present disclosure, by determining errors in measured bio-component data for predicting the growth of children and adolescents to generate refined input data, it is possible to increase the accuracy of growth prediction of children and adolescents.

According to various embodiments of the present disclosure, by determining errors in measured bio-component data for predicting the growth of children and adolescents to generate refined input data, it is possible to increase the accuracy of solutions necessary for children and adolescents.

The effects of the present disclosure are not limited to the above-mentioned effects, and other effects that are not mentioned may be obviously understood by those skilled in the art from the following description.

### BRIEF DESCRIPTION OF THE DRAWINGS

FIG. 1 is a view illustrating a structure of a growth prediction system structure according to an embodiment of the present disclosure.

FIG. 2 is a diagram illustrating the configuration of a growth prediction and solution generation device according to an exemplary embodiment of the present disclosure.

FIG. 3 is a diagram illustrating a data preprocessing unit of FIG. 2.

FIG. 4 is a diagram illustrating a predicted height and target height by growth stage according to an exemplary embodiment of the present disclosure.

FIG. 5 is a diagram illustrating the growth in obesity and normal weight for each growth stage of a boy according to an exemplary embodiment of the present disclosure.

FIG. 6 is a diagram illustrating the growth in obesity and normal weight by growth stage of a girl according to an exemplary embodiment of the present disclosure.

FIG. 7 is a diagram illustrating a configuration of a neural network for performing growth prediction and solution provision according to an exemplary embodiment of the present disclosure.

FIG. 8 is a diagram illustrating a first neural network model according to an exemplary embodiment of the present disclosure.

FIG. 9 is a diagram illustrating a second neural network model according to an exemplary embodiment of the present disclosure.

FIGS. 10 to 12 are diagrams illustrating a method of preprocessing bio-component measurement data for growth prediction according to an exemplary embodiment of the present disclosure.

FIG. 13 is a diagram illustrating bio-component data of a subject according to an exemplary embodiment of the present disclosure.

FIG. 14 is a diagram illustrating data concatenating bio-component data and identification data of a subject according to an exemplary embodiment of the present disclosure.

### Best Model

Hereinafter, detailed embodiments of the present disclosure will be described with reference to the accompanying drawings. The following detailed descriptions are provided to help a comprehensive understanding of methods, devices and/or systems described herein. However, the embodiments are described by way of examples only and the present disclosure is not limited thereto.

In describing exemplary embodiments of the present disclosure, when it is decided that a detailed description of a well-known technology related to the present disclosure may unnecessarily obscure the gist of the present disclosure, the detailed description will be omitted. Further, the following terminologies are defined in consideration of the functions in the present disclosure and may be construed in different ways by the intention of users and operators. Therefore, the definitions thereof should be construed based on the contents throughout the specification.

The terms used in the detailed description is merely for describing the embodiments of the present disclosure and should in no way be limited. Unless explicitly used otherwise, expressions in a singular form include the meaning in a plural form.

In the present description, expressions such as “include” or “comprise” are used to refer to certain features, numbers, steps, operations, components, or some or a combination thereof, and should not be construed to preclude the presence or addition of one or o more other features, numerals, steps, operations, components other than those described, or some or a combination thereof.

In addition, terms ‘first’, ‘second’, A, B, (a), (b), and the like, will be used in describing components of exemplary embodiments of the present disclosure. These terms are used only to differentiate the components from other components. Therefore, the nature, times, sequence, etc. of the corresponding components are not limited by these terms.

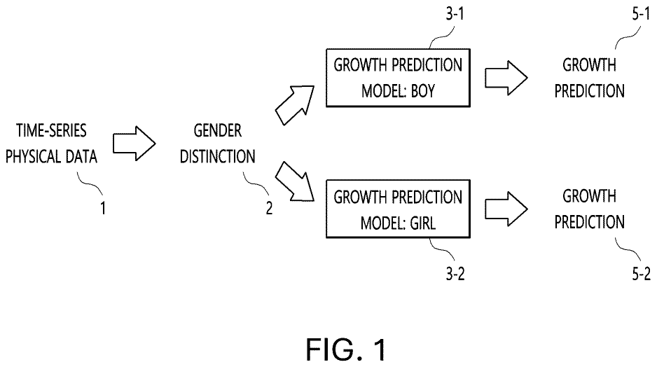

FIG. 1 is a view illustrating a structure of a growth prediction system structure according to an embodiment of the present disclosure.

Referring to FIG. 1, a growth prediction system of the present embodiment may receive subject's time-series physical data 1 and then distinguish a subject's gender 2 based on the received physical data 1.

Based on the distinguished gender, when the subject is a boy, growth prediction 5-1 may be performed through a growth prediction model 3-1, and when the subject is a girl, growth prediction 5-2 may be performed through a growth prediction model 3-2 that is different from the growth prediction model 3-1 used for the boy.

In the case of boys and girls, the growth rate by growth stage may be different. For example, as described below, boys and girls may each enter a rapid growth stage at different times, and thus the growth rate, the generation of solutions considering the growth rate, etc., may be different. Therefore, it is desirable to perform growth prediction using a model trained through different learning data based on the gender of the subject.

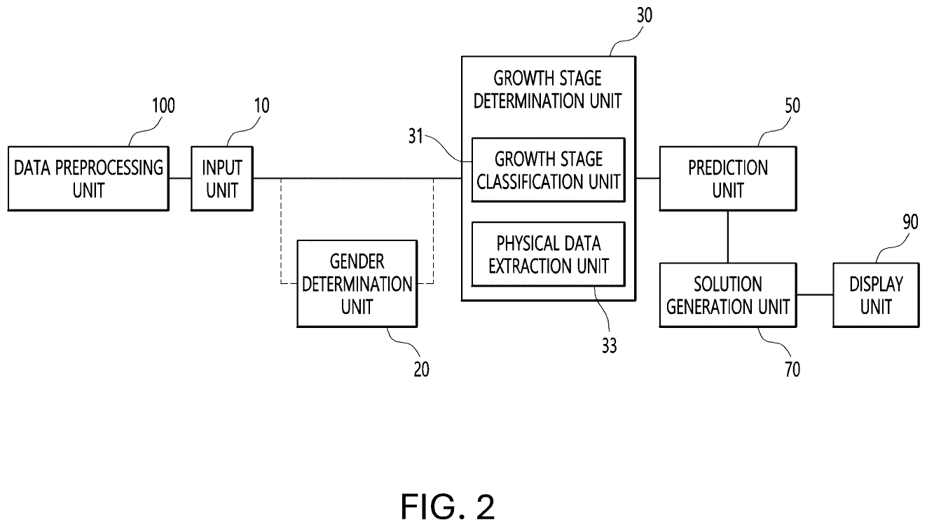

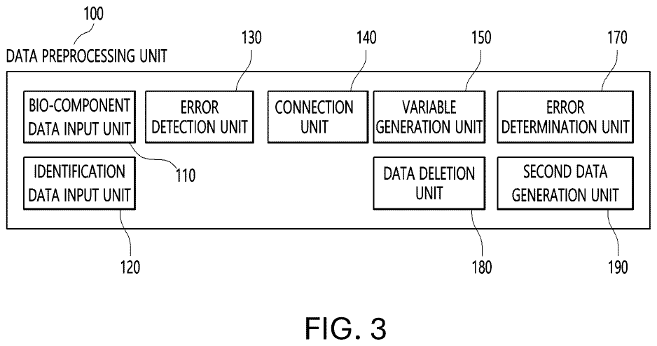

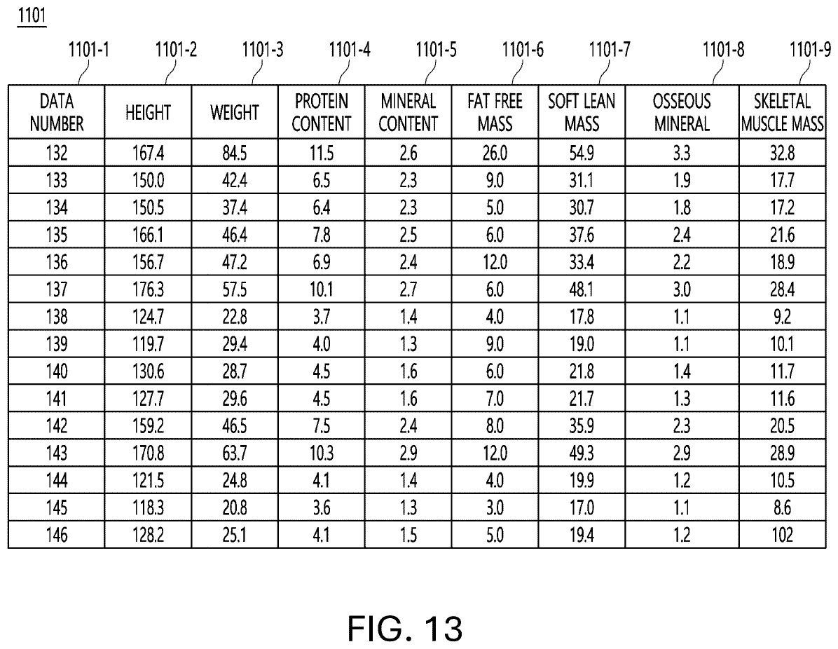

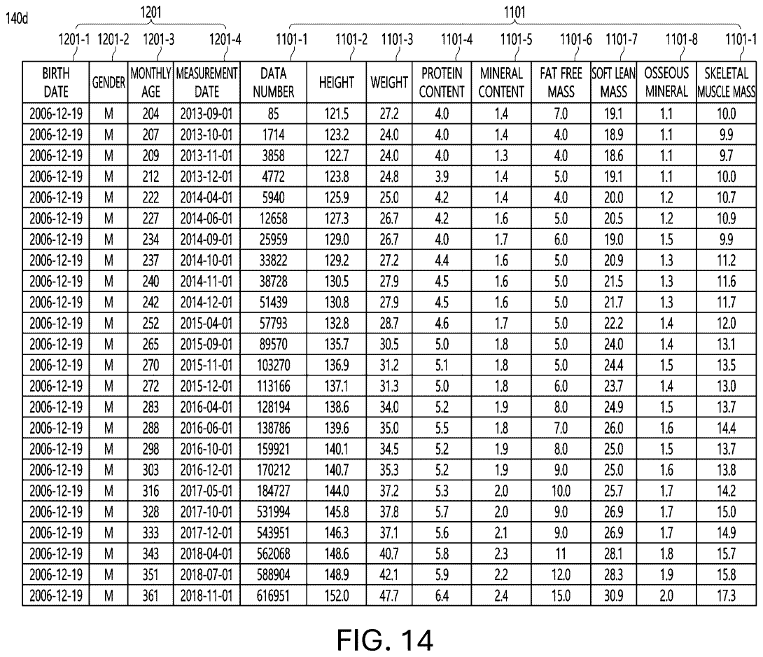

FIG. 2 is a diagram illustrating the configuration of a growth prediction and solution generation device according to an exemplary embodiment of the present disclosure, FIG. 3 is a diagram illustrating a data preprocessing unit of FIG. 2, FIG. 13 is a diagram illustrating bio-component data of a subject according to an exemplary embodiment of the present disclosure, and FIG. 14 is a diagram illustrating data concatenating bio-component data and identification data of a subject according to an exemplary embodiment of the present disclosure.

Hereinafter, the description will be made with reference to FIGS. 2, 3, 13, and 14.

More specifically, the growth prediction and solution generation device according to an exemplary embodiment of the present disclosure may include a data preprocessing unit 100, an input unit 10, a gender determination unit 20, a growth stage determination unit 30, a prediction unit 50, a solution generation unit 70, and a display unit 90.

Through the input unit 10, the growth prediction and solution generation device may receive the time-series physical data of the subject.

The physical data of the subject may include data (identification data) that may identify the subject and the bio-component data of the subject.

As an example, the identification data may include data for identifying a subject such as grade (or age), gender, and height, and the bio-component data may include data such as weight, protein, mineral content, body fat, body water, soft lean mass, fat free mass (body fat mass), bone tissue, skeletal muscle mass, body mass index (BMI), basal metabolic rate, neck circumference, chest circumference, abdominal circumference, thigh circumference, arm circumference, and hip circumference.

More specifically, referring to FIGS. 13 and 14, identification data 1201 of a subject may include a birth date 1201-1, a gender 1201-2, a monthly age 1201-3, and a measurement date 1201-4.

In addition, a bio-component data 1101 of the subject may include a data number 1101-1, a height 1101-2, weight 1101-3, a protein content 1101-4, a mineral content 1101-5, a fat free mass 1101-6, a soft lean mass 1101-7, osseous mineral 1101-8, and a skeletal muscle mass 1101-9.

The physical data is only an example to help understand the present disclosure, and the present embodiment is not limited thereto. Of course, the types of information constituting the physical data may be changed in various ways according to the embodiment.

Meanwhile, an exemplary embodiment of the present disclosure may include the data preprocessing unit 100 that determines an error in the physical data of the subject to generate refined data.

Since the bio-component data 1101 may undergo rapid changes depending on the growth stage due to the physical characteristics of children and adolescents, errors (errors in the measurement process) may occur in other bio-component data measurement processes such as a measurement method, a measurement time, and a measurement environment.

In addition, errors (data transmission errors) may occur in the process of transmitting or storing the measured bio-component data.

Therefore, the data preprocessing unit 100 of the present embodiment generates a virtual variable (hereinafter referred to as a first variable) based on the measured bio-component data 1101, and then compares the first variable with a value of any one of the bio-component data to determine an error in the measurement process and a data transmission error in the measured bio-component data 1101, thereby generating refined data.

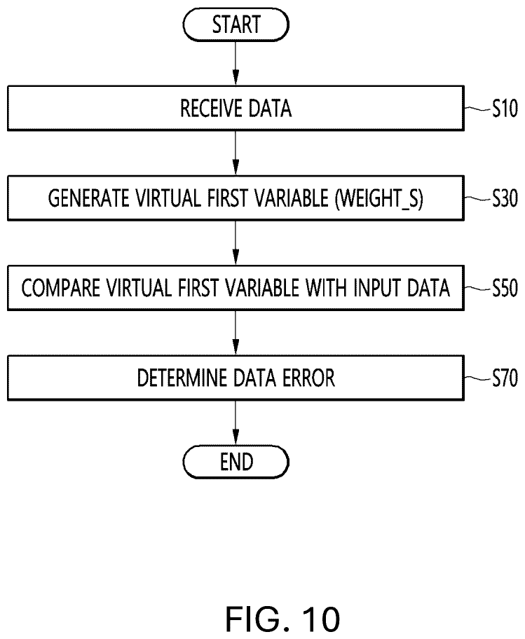

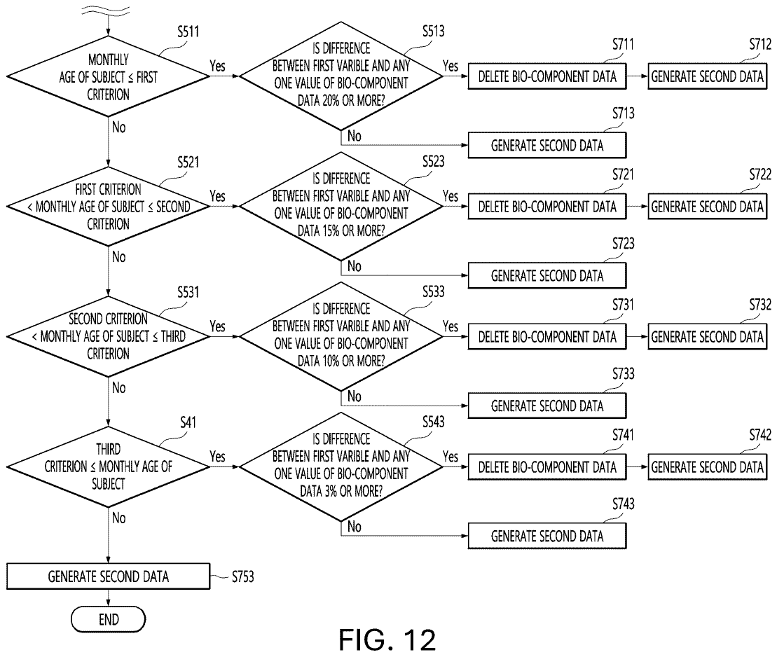

This will be described with reference to FIGS. 10 to 12 below.

Meanwhile, the data input to the input unit 10 may include the time-series physical data of the subject.

The time-series physical data may be continuous data or discontinuous data, but may be data included in at least one period corresponding to the growth stage of children and adolescents.

More specifically, the collection period and the number of times of collections of the time-series physical data of the subject may vary.

For example, a first subject may have measured physical data from ages 8 to 12, which is part of the children and adolescents period, and a second subject may have irregularly measured physical data such as ages 8, 10 to 12, and 15.

In addition, a third subject may have physical data measured multiple times during a certain period (period corresponding to any one of the growth stages of children and adolescents), while a fourth subject may have physical data measured only once during a certain period (period corresponding to any one of the growth stages of children and adolescents).

As described above, depending on the collection period and the number of times of collections, the physical data of the subject may be included in two or more of the growth stages of children and adolescents (the first subject, the second subject), but may not be included therein (the third subject and the fourth subject).

As in the third subject, when there is the physical data measured multiple times during one of the plurality of growth stages, the growth stage determination unit 30 may classify the growth stage in which the physical data of the third subject is included through a growth stage classification unit 31, and extract the physical data through a physical data extraction unit 33. Here, the extracted physical data may generally include all of the physical data of the third subject input through the input unit 10.

However, as in the fourth subject, when the physical data of the subject corresponds to only any one of the plurality of growth stages, and there is physical data measured only once during that period, physical data corresponding to an arbitrary period may further be generated before the physical data of the fourth subject is input to the growth stage determination unit 30.

More specifically, time-series physical data of a subject corresponding to an arbitrary period may be generated based on the time-series physical growth data of the plurality of sample subjects in which the input physical data of the subject have been previously stored.

As an example, the physical data may be generated based on a distribution model (similarity) between the input physical information of the subject and the pre-stored time series physical growth data of the plurality of sample subjects, or the physical data may be generated based on a Bayesian inference model (conditional probability).

The growth stage determination unit 30 may classify the growth stage into any one of the plurality of growth stages in the growth stage classification unit 31 based on the physical data of the subject input through the input unit 10 and extract physical data corresponding to the classified growth stage in the physical data extraction unit 33.

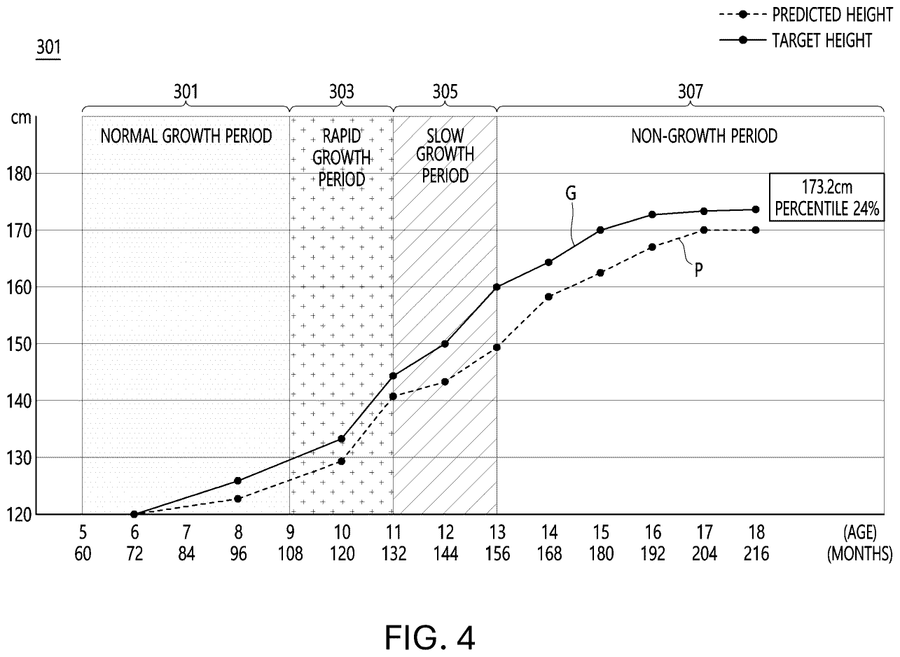

FIG. 4 is a diagram illustrating a predicted height and target height by growth stage according to an exemplary embodiment of the present disclosure.

In FIG. 4, an x-axis represents age and months, and a y-axis represents height (cm). A dotted line P below represents a predicted growth rate of the evaluation, and a solid line G above represents a target growth rate of the evaluation subject.

Describing the growth stages of children and adolescents described above with reference to FIG. 4, the growth stage may include a normal growth period 301, a rapid growth period 303, a decelerated growth phase 305, and a non-growth period 307.

Each growth stage may be classified by the degree of growth, and the height grown each year varies depending on each growth stage, and the actual height grown even in the same growth stage may vary depending on the growth type.

The normal growth period 301 generally refers to the period before puberty when secondary sexual characteristics appear. The children and adolescents corresponding to this period generally have open growth plates. As a result, depending on the growth environment, in the case of a short height growth type, a height generally grows by 4 to 5 cm per year, and in the case of a tall growth type, a height grows in the range of 6 to 7 cm per year.

The rapid growth period 303 is a period in which secondary sexual characteristics begin to appear. In women, a breast swells and a lump appears, and in men, the testicles grow larger, pubic hair begins to grow, and a voice break appears where voice changes. The rapid growth period 303 generally lasts about 2 to 3 years after the normal growth period 301, and a height grows in the range of 7 to 10 cm per year on average.

The decelerated growth period 305 refers to a period in which the secondary sexual characteristics are completed. During this period, in the case of women, the secondary sexual characteristics may be clearly identified starting from menarche, and in the case of men, the secondary sexual characteristics may be clearly identified through pubic hair, voice change, and armpit hair. In the decelerated growth period 305, the growth rate drops rapidly compared to the rapid growth period 303. The decelerated growth period 305 generally lasts about 2 to 3 years, and a height grows in the range of 5 to 6 cm per year on average, and does not naturally grow any further. The growth plate begins to close little by little after the rapid growth period 304, and closes approximately 50% about 6 months after entering the decelerated growth period 305.

The non-growth period 307 refers to a period in which the growth plate has closed, as a period in which the growth period has not completely ended but natural height growth has become difficult. Generally, women enter the non-growth period 307 about 1 year and 6 months to 2 years after menarche, and men enter the non-growth period 307 about 1 year and 6 months to 2 years from the time hair begins to appear in armpits. In the non-growth period 307, the growth plate closes and the natural growth stops, but by changing bad lifestyle habits and improving a physical function through customized exercise, posture correction, and nutrient intake, etc., a height may grow in the range of about 1 to 3 cm.

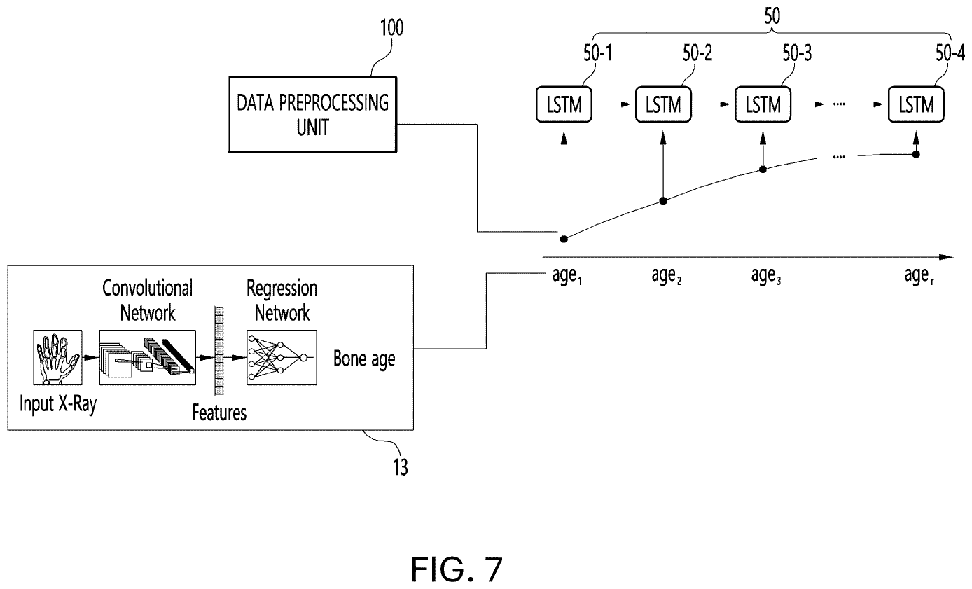

Meanwhile, the prediction unit 50 is a prediction model and may be implemented with artificial intelligence in a recursive neural network (RNN) structure so that it may use not only current values but also time series values. For example, the prediction model may be implemented with an architecture such as Long Short Term Memory (LSTM), or Gated Recurrent Units (GRU) that is the RNN. Of course, in addition to this, conventional various artificial intelligence architectures may be applied to the prediction model of this embodiment, which will be described in detail with reference to FIGS. 7 to 9 described later.

The solution generation unit 70 may generate a growth management solution based on the physical data of the subject corresponding to the classified growth stage.

More specifically, when the subject corresponds to the normal growth period 301, a solution for increasing the growth prediction value of the subject may be provided. The growth prediction value is a value corresponding to the y-axis in FIG. 4, and the solution for increasing the growth prediction value may be provided to the subject through various solution display units 90 for increasing the expected target value of the y-axis.

Meanwhile, examples of solutions provided through the display unit 90 may include a current height, a predicted height, an obesity level, a fat free mass, a skeletal muscle mass, a protein content, a mineral content, an amount of sleep, an amount of exercise, nutritional information, lifestyle habits, posture, etc. Each indicator may be expressed step by step as caution, normal, good, etc., based on a preset range, or may also be expressed as a level.

The current state, customized solutions, precautions, etc., for each indicator may be displayed. The current state may be displayed step by step or level based on the target value. The customized solutions may include contents for adjustment of protein, mineral content, body fat, body water, soft lean mass, fat free mass, bone tissue, skeletal muscle mass, body mass index (BMI), basal metabolic rate, etc., to reach the current target value based on the input physical data.

The precautions may include contents for adjustment of the current insufficient amount of protein, mineral content, body fat, body water, soft lean mass, fat free mass, bone tissue, skeletal muscle mass, body mass index (BMI), basal metabolic rate, etc., that are currently lacking, based on the input physical data.

In addition, when the subject corresponds to the rapid growth period 303, a solution for increasing the growth prediction value of the rapid growth period 303 of the subject may be provided. The growth prediction value is a value corresponding to the y-axis in FIG. 2, and the solution for increasing the growth prediction value may be provided to the subject through various solution display units 90 for increasing the expected target value of the y-axis.

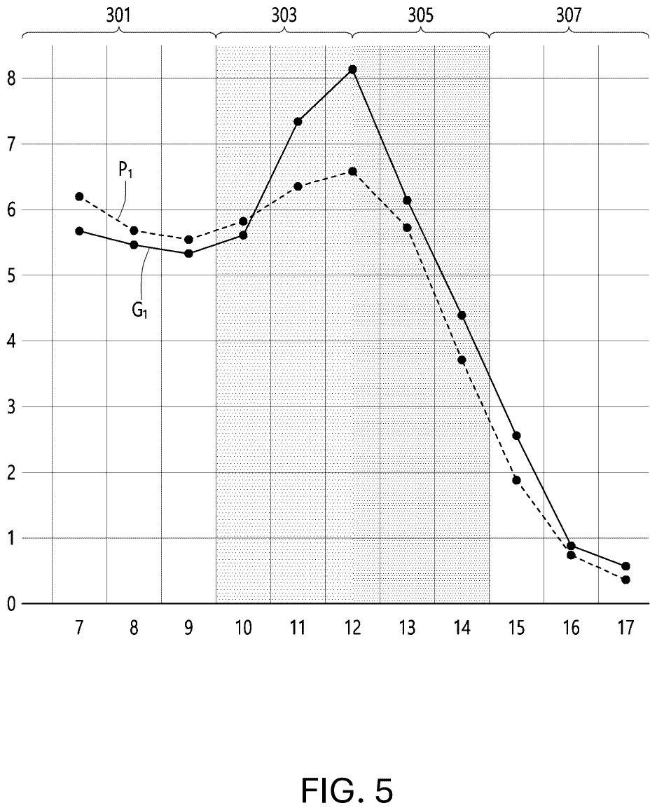

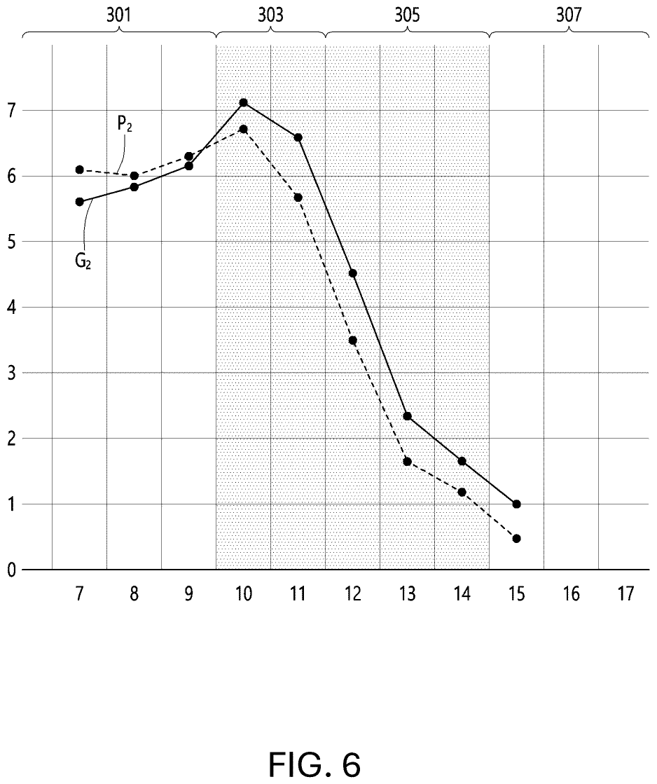

FIG. 5 is a diagram illustrating the growth in obesity and normal weight for each growth stage of a boy according to an exemplary embodiment of the present disclosure, and FIG. 6 is a diagram illustrating the growth in obesity and normal weight by growth stage of a girl according to an exemplary embodiment of the present disclosure.

In FIG. 5, an x-axis refers to the age, and a y-axis refers to the growth rate (cm). A solid line G1 represents the growth rate by growth stage in the case of normal weight for boys, and a dotted line Pl represents the growth rate for each growth stage in the case of obesity for boys.

In FIG. 6, an x-axis refers to the age, and a y-axis refers to the growth rate (cm). A solid line G2 represents the growth rate for each growth stage in the case of normal weight for girls, and a dotted line P2 represents the growth rate for each growth stage in the case of obesity for girls.

This will be described with reference to FIGS. 5 and 6 below.

Obesity is not a simple increase in weight, but is overweight accompanied by excessive accumulation of fat tissue in the body or a disease that is accompanied by metabolic disorders caused by the overweight. The obesity in children and adolescents medically refers to a case in which the body weight is 20% or more than a standard weight for each height in an age group from infancy to puberty.

The obesity in the infancy usually disappears after a first birthday of a child as the movement and activity of the child become more active. However, in the case of some children, obesity persists, and there are many cases where weight returns to normal but obesity recurs at school age.

75% to 80% of obesity in children and adolescents transitions to adult obesity. In addition, obesity inhibits a secretion of growth hormones. Especially, in the case of girls, puberty is accelerated and the period of growth potential is shortened, so growth is hindered or precocious puberty is caused.

Therefore, it is necessary to provide systematic solutions to predict obesity and prevent obesity when obesity is predicted for school-age children and adolescents who are prone to obesity.

Meanwhile, obesity in children and adolescents may be divided into simple obesity for which the exact cause is not known and symptomatic obesity caused by a special causative disease, and more than 99% of childhood obesity is simple obesity.

Both boys and girls with simple obesity tend to have average height or be slightly taller than those of the same age group (a plurality of sample subjects) in the normal growth period 301, but tend to be shorter or have a lower growth rate than those of the same age group (a plurality of sample subjects) after the rapid growth period 303.

In summary, obesity in children and adolescents may be understood as a group of diseases that are accompanied by overweight or metabolic disorders resulting from a wide variety of causes. Referring to FIGS. 5 and 6, boys and girls tends to generally have average or slightly higher growth rates than the plurality of sample subjects of the same age group in the normal growth period 301, but tends to have a lower growth rate than the plurality of sample subjects of the same age group after the rapid growth period 303.

Therefore, in order to more accurately predict the obesity and generate the solution based on the growth stage, the present embodiment may classify the gender of the subject through the gender determination unit 20 based on the physical data of the subject input through the input unit 10 and then classify the growth stage in the growth stage determination unit 30 based on the classified gender, and extract the physical data, and then generate the solution in the solution generation unit 70 by considering the gender and growth stage of the subject.

More specifically, when boys and girls commonly correspond to obesity, it can be seen that the growth prediction value in the rapid growth period 303 is lower than in the normal case. Therefore, when the subject corresponds to the rapid growth period 303, the solution for increasing the growth prediction value may be provided, and when the subject corresponds to the normal growth period 301, it may include information on adjustment of indicators that may alleviate, particularly, abnormal increases in sex hormones, including the physical data that are taken into account.

In addition, when the subject corresponds to the slow growth period 305, a solution for adjusting the period of the slow growth period 305 of the subject may be provided. The growth stage period adjustment may be divided into cases where the physical data of the subject is located at the beginning of the slow growth period 305 and cases where the physical data of the subject is located in the mid to late stage of the slow growth period 305 among the growth stages classified based on the input physical data of the subject.

The standard for distinguishing between the beginning and the mid to late stage of the above-mentioned slow growth period 305 may be divided based on a predetermined range corresponding to the slow growth period 305 from the rapid growth period 303 with respect to the x-axis in FIGS. 4 to 6. Alternatively, based on whether the secondary sexual characteristics are completed based on the input physical information of the subject, when the secondary sexual characteristics are not completed, it may be classified as the beginning of the slow growth period 305, and when the secondary sexual characteristics are completed, it may be divided as the mid to late part of the decelerated growth period 305.

Preferably, it is possible to determine whether the secondary sexual characteristics have been completed based on the input physical information of the subject to determine whether the current physical information of the subject is located at the beginning or mid-to-late part of the decelerated growth period 305, and when it is not possible to determine whether the secondary sexual characteristics have been completed based on the input physical information of the subject, it is possible to determine whether the physical information of the subject is located at the beginning or mid-to-late part of the decelerated growth period 305 based on the predetermined range corresponding to the decelerated growth period 305 from the rapid growth period 303.

Meanwhile, when the input physical data of the subject is located at the beginning of the decelerated growth period 305, a period adjustment solution for delaying the entry into the decelerated growth period 305 may be provided.

As described above, the secondary sex characteristics are being completed when transitioning from the rapid growth period 303 to the decelerated growth period 305, so it is possible to provide a solution for delaying the time when the secondary growth is completed, and in FIGS. 4 to 6, various solutions for moving the range of the x-axis corresponding to the decelerated growth period 305 to the right may be provided to the subject through the display unit 90. In this case, the range of decelerated growth period 305 may increase depending on the physical information of the subject, or may decrease as the rapid growth period 303 increases.

Meanwhile, when the input physical data of the subject is located at the mid to late part of the decelerated growth period 305, the period adjustment solution for increasing the decelerated growth period 305 may be provided.

As described above, the decelerated growth period 305 refers to the time when a growth plate of the subject closes. Generally, about 50% of the growth plate closes 6 months after entering the decelerated growth period 305, and when the growth plate closes and the natural growth stops, the non-growth period 307 is entered. In this case, a solution for increasing the period of decelerated growth period 305 may be provided. In other words, various solutions for widening the range of the x-axis corresponding to the decelerated growth period 305 in FIGS. 4 to 6 may be provided to the subject through the display unit 90.

When the above-described subject corresponds to the normal growth period 301, it may include, especially, contents on adjustment of indicators that may alleviate the degree that the growth plate closes, including the physical data to be considered.

In addition, when the subject corresponds to the non-growth period 307, a solution for improving physical functions through lifestyle habits, customized exercise, posture correction, nutrient intake, etc., based on the input physical data of the subject may be provided.

In the non-growth period 307, the growth plate closes and the natural growth stops, so the solutions for improving the physical functions through the lifestyle habits, the customized exercise, the posture correction, etc., based on body weight, body fat, body water, soft lean mass, skeletal muscle mass, body mass index (BMI), basal metabolic rate, neck circumference, chest circumference, abdominal circumference, thigh circumference, arm circumference, and hip circumference, etc., of the subject may be provided or the solution for improving the physical functions through the nutrient intake, etc., based on protein, mineral content, bone tissue (bone density), etc., may be provided.

Meanwhile, in order to more accurately predict the obesity and provide the solution in the present embodiment, the gender of the subject may be classified through the gender determination unit 20, and the growth stage classification unit 31 may set the timing of the rapid growth stage differently based on the classified gender.

As described above, this is because the entry time into the rapid growth stage may be different for boys and girls. The physical data of the subject corresponding to the rapid growth stage considering the gender output from the physical data extraction unit 33 is input to the prediction unit 50 to output the prediction value for obesity.

When the subject is predicted to be obese and the gender is male, a solution for increasing the growth prediction value of the subject in the rapid growth period 303 may be provided, which is as described above.

When the subject is predicted to be obese and the gender is woman, a solution for increasing the period of the rapid growth period 303 may be provided.

In more detail, referring to FIGS. 5 and 6, in the case of woman, the difference in the growth prediction value in the rapid growth period 303 is smaller than that of men, but as it transitions from the rapid growth period 303 to the decelerated growth period 305, the growth rate of height decreases significantly with age.

For example, in FIG. 5, it can be seen that, in the case of a man having normal weight, the difference in the growth prediction value in the rapid growth period 303 is clearly different from the difference in the growth prediction value in the case of the normal weight G1 and the case of the obesity P1. Therefore, in the case of the obese man, there is a need to provide the solution for increasing the growth prediction value in the rapid growth period 303.

In addition, as an example, in FIG. 6, a woman having normal weight grows 7 cm in height as she grows from 9 to 10 years old, and grows about 6.5 cm in height as she grows from 10 to 11 years old.

In contrast, it can be seen that an obese woman grows approximately 6.7 cm in height from 9 to 10 years old, and approximately 5.7 cm in height from 10 to 11 years old. In FIG. 5, it can be seen that the difference in growth prediction value is relatively small when compared to the case of man.

Therefore, in the case of the obese women, it is necessary to provide the solution for increasing the period of the rapid growth period 303 to reduce the decrease range of the growth rate that occurs when transitioning from the rapid growth stage to the decelerated growth stage.

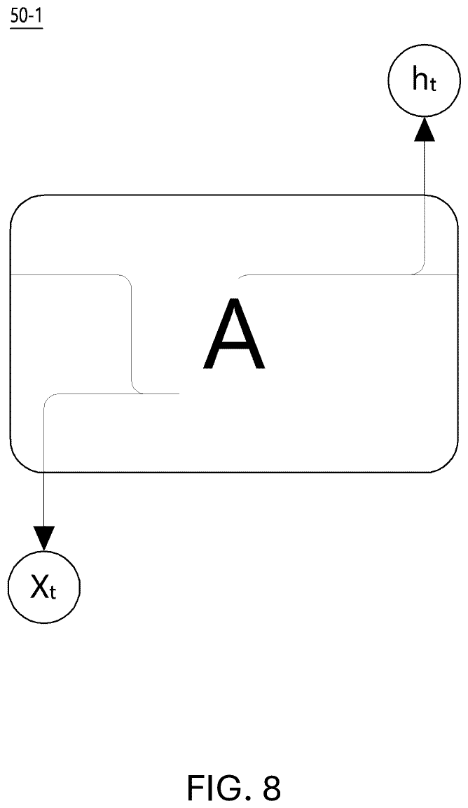

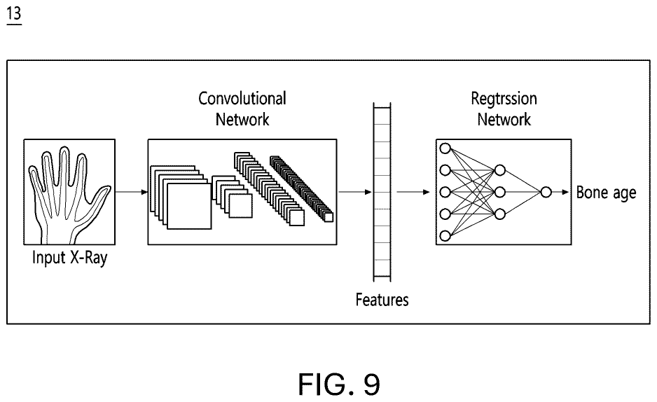

FIG. 7 is a diagram illustrating a configuration of a neural network for performing growth prediction and solution provision according to an exemplary embodiment of the present disclosure, FIG. 8 is a diagram illustrating a first neural network model according to an exemplary embodiment of the present disclosure, and FIG. 9 is a diagram illustrating a second neural network model according to an exemplary embodiment of the present disclosure.

This will be described with reference to FIGS. 7 to 9 below.

An exemplary embodiment of the present disclosure may include a first model 50 and a second model 13, and a pipeline may be built in which at least some of the output of the second model 13 is input to the first model 50.

More specifically, the first model 50 is a model that trains physical data corresponding to at least one of the plurality of growth stages as training data based on the time-series physical data for the plurality of sample subjects.

In the growth stage, the rapid growth period 303, which is the time when growth slowdown due to obesity begins, may be adopted, but as described above, any one or more of the plurality of growth stages may be adopted to more accurately predict obesity.

The first model 50 includes LSTM neural networks 50-1, 50-2, 50-3, and 50-4 for training time-series data, and trains the LSTM neural networks 50-1, 50-2, 50-3, and 50-4 using past physical information of the plurality of sample subjects. The physical data of the current subject may be input to the trained LSTM neural networks 50-1, 50-2, 50-3, and 50-4 to output the predicted growth rate and the solution considering the growth rate.

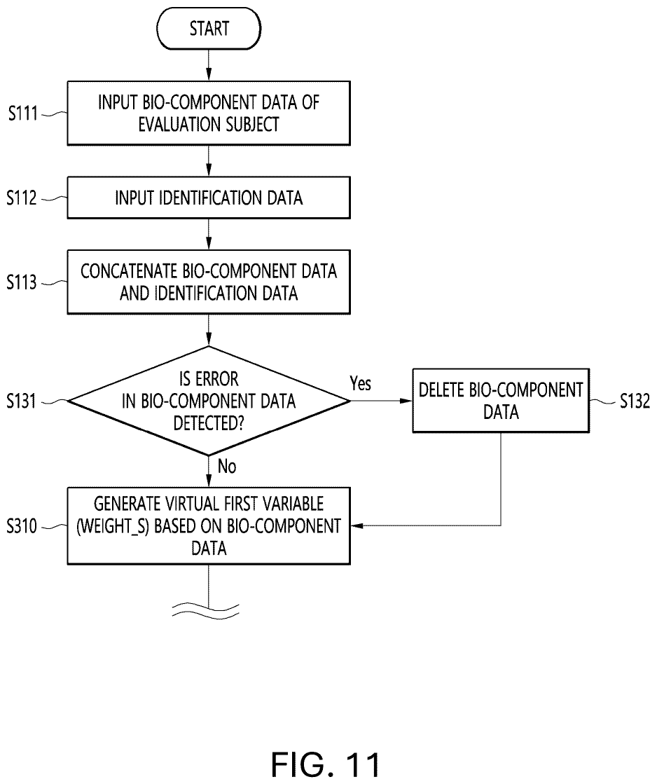

The physical data of the subject may mean the refined data that has gone through an error determination process through the data preprocessing unit 100, which will be described in more detail later with reference to FIGS. 10 and 11.

The LSTM neural networks 50-1, 50-2, 50-3, and 50-4 are trained using at least any one of the physical data of the plurality of sample subjects as a default. For example, for height, training is performed with annual height data during an arbitrary period or specific growth stage, and the prediction for the next year is made and compared with actual data. By this comparison, the training set is trained as it moves into the future at random periods or in units of specific growth stages.

In addition, the LSTM neural networks 50-1, 50-2, 50-3, and 50-4 may be trained for each growth stage. Therefore, the normal growth period 301, the rapid growth period 303, the decelerated growth period 305, and the non-growth period 307 may each be trained with the past physical data of the corresponding growth stage.

The physical data of the subject for the training may mean the refined data that has gone through an error determination process through the data preprocessing unit 100, which will be described in more detail later with reference to FIGS. 10 and 11.

Meanwhile, illustratively, in this embodiment, the time series physical information on the plurality of sample subjects is sequentially input as the training data according to age or arbitrary period, and the calculation result of the prediction value at the past point in time or growth rate may be transmitted to the growth rate prediction at the next age or arbitrary period.

Therefore, the LSTM neural networks 50-1, 50-2, 50-3, and 50-4 may not only predict the growth rate based on the current physical data, but also train the extent to which prediction results 50-1, 50-2, 50-3, and 50-4 by various indicators at the past point in time affects the current growth rate prediction, so items that have a significant impact on the change in the growth rate depending on age or arbitrary period among the indicators may be extracted and reflected in the growth rate prediction.

In addition, for time series learning, it is necessary to secure the physical data of the plurality of sample subjects at regular intervals. However, as described above, it may be difficult to regularly obtain the physical data of the plurality of sample subjects depending on age or arbitrary period, so it can be used by removing outlier physical data or non-continuous physical data for each unit period and normalizing it in time.

Meanwhile, the second model 13 may derive bone maturity (age) from a carpal image using a convolution neural network trained with bone maturity data of a subject as training data.

More specifically, the convolutional neural network may include a plurality of convolution layers that creates a feature map for features in an image to be analyzed among the carpal images and a pooling layer where sub-sampling is performed between the plurality of convolutional layers to extract features at different levels for an area to be analyzed, may be inferred probabilistically through an activation function, or may derive the bone maturity through weight learning between nodes through regression analysis.

The bone maturity extracted through the second model 13 may be input to the LSTM neural networks 50-1, 50-2, 50-3, and 50-4 along with at least some of the physical data of the subject to increase the accuracy of predicting the growth rate of the subject, thereby further increasing the accuracy of predicting the obesity.

FIGS. 10 to 12 are diagrams illustrating a method of preprocessing bio-component measurement data for growth prediction according to an exemplary embodiment of the present disclosure.

As described above, when predicting the growth of the subject (children and adolescents) or providing the solution through the growth prediction of the subject (children and adolescents), the input physical data may have the error in the measurement process or the error in the data transmission.

Therefore, in order to more accurately predict the growth prediction or providing the solution through the growth prediction of the subject, it is preferable to input the refined data that has gone through the process of detecting and determining the above-described error data to the input unit 10.

In addition, the physical data of the plurality of sample subjects for training the prediction model of the above-described prediction unit 50 may also use the refined data that has gone through the process of detecting and determining the above-described error data.

Hereinafter, the process of detecting and determining errors in physical data will be described with reference to FIGS. 3, 10 to 14.

An exemplary embodiment of the present disclosure may receive the physical data (S10) to detect or determine the error in the physical data of the subject, particularly the error in the bio-component data, and then generate the virtual first variable based on the input physical data (S30). The generated virtual first variable may be compared with the physical data received in the step (S10) (S50) to determine an error in the received physical data (S70).

More specifically, the physical data of the subject may include the identification data 1201 and the bio-component data 1101 as illustrated in FIG. 14, and the preprocessing unit 100 may receive the bio-component data 1101 of the subject (S111) through a bio-component data input unit 110, and receive the identification data 1201 of the subject through an identification data input unit 120 (S112). The received bio-component data 1101 and the identification data 1201 may be generated as first data 140d that is concatenated through the connection unit 140.

When it is determined that the error is detected in the bio-component data 1101 input through the process (S131) of detecting the error in the bio-component data through an error detection unit 130 (S131: Yes), at least some of the bio-component data constituting the concatenated data 140d may be deleted (S132).

For example, when at least any one of the plurality of items 1101-1 to 1101-9 that constitute the input bio-component data 1101 includes a ‘Nan’ value, the data preprocessing unit 100 of the present embodiment may delete the bio-component data 1101 through a data deletion unit 180. In this case, all items (rows including the measurement date) measured at the measurement date including the ‘Nan’ value based on the measurement date (1201-4) of the identification data may be deleted.

Alternatively, when the monthly age is 30 months or less based on the monthly age 1201-3 of the identification data, all items (rows including the measurement date) measured at the measurement date that include the ‘Nan’ value may be deleted. In more detail, when the monthly age is 30 months or less, it is a period when rapid growth of infants and toddlers occurs, and may include many errors during the measurement, so the reliability of the bio-component data is relatively lower than that of bio-component data with a high monthly age. Therefore, when the bio-component data includes the ‘Nan’ value as described above when the monthly age is 30 months or less, all items measured at the measurement date that include the ‘Nan’ value may be deleted.

The deletion of the bio-component data described above is exemplary, and when there is an error (the Nan value) in the items that constitute the bio-component data by various criteria, the error may be detected and deleted.

When no error is detected in bio-component data 1101 (S131 No), a virtual first variable may be generated (S310) through a variable generation unit 150.

The first variable may be generated as the sum of the fat free mass 1101-6, the soft lean mass 1101-7, and the osseous mineral 1101-8. For reference, the soft lean mass 1101-7 may be defined as the sum of the body water content, the protein content 1101-4, and the non-osseous mineral content.

As described above, by generating the virtual first variable (S310) and then comparing (S50) the first variable with the data input in the steps (S111 and S112), the error in the input bio-component data 1101 may be determined.

More specifically, first, it may be determined whether the monthly age 1201-3 of the subject in the identification data 1201 of the subject corresponds to a preset criterion, and then, it may be determined whether the bio-component data is deleted based on the degree of difference between the first variable and the weight 1101-3 among the bio-component data 1101 based on the preset standard, thereby generating the refined data (second data).

The bio-component data 1101 input in the step (S111) may be obtained by being measured through bioelectrical impedance analysis (BIA), which is a method of measuring the composition of the body based on the speed at which the current passes through the body.

Since the bio-component data measured in this way has a large range of changes in body compositions as the subject gets younger, and there is a high possibility of errors occurring during the measurement process, the method of preprocessing bio-component measurement data of the present embodiment determines whether the monthly age of the subject falls within multiple preset criteria, and then determines errors in the bio-component measurement data under different conditions according to each preset criterion.

For example, in the present embodiment, when the difference between the generated first variable and weight 1101-3 is greater than the preset range, it is determined that an error has occurred in the measurement value generating the first variable, and the refined data may be generated through the process of deleting a row including a measurement value generating the first variable from concatenated data 140d.

As described above, the reason for comparing the virtual first variable and the weight data is that theoretically, the weight data 1101-3 is identical to the first variable, but the difference between the weight data 1101-3 and the first variable may occur due to various variables. Therefore, when the difference between the weight data 1101-3 and the first variable is outside the preset range, the measurement values of the row including the measurement value generating the first variable are determined as errors and deleted.

Hereinafter, the process of determining the error in the bio-component measurement data described above through an error determination unit 170 will be described.

After the first variable is generated in the step (S310), it may be determined whether the monthly age of the subject is below the first criterion, and the first criterion may be set to 60 months (S511).

When the monthly age of the subject is less than or equal to the first criterion (S511: Yes), it may be determined whether the difference between the first variable and any one value of the bio-component data is 20% or more (S513). Any one value of the bio-component data may be set to the weight data 1101-3.

When the difference between the first variable and the weight data 1101-3 is 20% or more (S513: Yes), the bio-component data may be deleted through the data deletion unit 180 (S711) and refined second data may be generated through a second data generation unit 190 (S712).

In addition, when the difference between the first variable and the weight data 1101-3 is not 20% or more (S513: No), the refined second data may be generated (S712) through the second data generation unit 190.

Meanwhile, when the monthly age of the subject exceeds the first criterion (S511: No), it may be determined whether the monthly age of the subject exceeds the first criterion and falls within the range of the second criterion (S521), and the second criterion may be set to 75 months.

When the monthly age of the subject corresponds between the first criterion and the second criterion (S511: Yes), it may be determined whether the difference between the first variable and any one value of the bio-component data is 15% or more (S523).

In the step (S523), the monthly age of the subject, which is the subject of the error determination, is higher than the monthly age of the subject, which is the subject of the error determination in the step (S513), so the range of changes in the body compositions is relatively small.

Therefore, the criterion (is the difference between the first variable and the weight data 15% or more?) for determining the error in the bio-component data input in the step (S523) may be set higher than the criterion (is the difference between the first variable and the weight data 20% or more?) for determining the error in the bio-component data input in the step (S513).

The fact that the criterion is set higher can mean that the range for determining the difference between the first variable and the weight data as an error in the present embodiment is narrower. That is, in the method of preprocessing bio-component measurement data of the present embodiment, the higher the monthly age of the subject, the higher the criterion for determining an error may be set.

When the difference between the first variable and the weight data 1101-3 is 15% or more (S523: Yes), the bio-component data may be deleted through the data deletion unit 180 (S721) and the refined second data may be generated through the second data generation unit 190 (S722).

In addition, when the difference between the first variable and the weight data 1101-3 is not 15% or more (S523: No), the refined second data may be generated through the second data generation unit 190 (S723).

Meanwhile, when the monthly age of the subject does not correspond between the first criterion and the second criterion (S511: No), it may be determined whether the monthly age of the subject exceeds the second criterion and falls within the range of the third criterion (S531), and the third criterion may be set to 100 months.

When the monthly age of the subject corresponds between the second criterion and the third criterion (S531: Yes), it may be determined whether the difference between the first variable and any one value of the bio-component data is 10% or more (S533).

When the difference between the first variable and the weight data 1101-3 is 10% or more (S533: Yes), the bio-component data may be deleted through the data deletion unit 180 (S731) and the refined second data may be generated through the second data generation unit 190 (S732).

In addition, when the difference between the first variable and the weight data 1101-3 is not 10% or more (S533: No), the refined second data may be generated through the second data generation unit 190 (S733).

Meanwhile, when the monthly age of the subject does not fall between the second and third criteria (S531: No), it can be determined whether the monthly age of the subject exceeds the third criterion (S541).

When the monthly age of the subject exceeds the third criterion (S511: Yes), it may be determined whether the difference between the first variable and any one value of the bio-component data is 3% or more (S543).

When the difference between the first variable and the weight data 1101-3 is 3% or more (S543: Yes), the bio-component data may be deleted through the data deletion unit 180 (S741) and the refined second data may be generated through the second data generation unit 190 (S742).

In addition, when the difference between the first variable and the weight data 1101-3 is not 3% or more (S543: No), the refined second data may be generated through the second data generation unit 190 (S743).

Meanwhile, when the monthly age of the subject does not exceed the third criterion (S541: No), there is an error in the monthly age data 1201-3 of the subject, or an error occurred in the process of determining whether the monthly age data 1201-3 of the subject is included in the range defined by the first to third criteria described above, so this process may end. Alternatively, the process of determining whether the monthly age of the subject is less than or equal to the first criterion (S511) may be repeatedly performed.

As described above, the generated second data is refined data that has gone through the process of determining an error based on the input bio-component data 1101 of the subject and identification data 1201, and may be used as the input data for the growth prediction or for the solution generation through the growth prediction. In this case, the second data may be used as data input to the input unit 10 of FIG. 2.

In addition, the second data may be used as growth prediction or training data for a model for growth prediction. In this case, the second data may be used as data input to the prediction model of FIG. 7.

Hereinabove, the present disclosure has been described with reference to exemplary embodiments. All exemplary embodiments and conditional illustrations disclosed in the present disclosure have been described to intend to assist in the understanding of the principle and the concept of the present disclosure by those skilled in the art to which the present disclosure pertains. Therefore, it will be understood by those skilled in the art to which the present disclosure pertains that the present disclosure may be implemented in modified forms without departing from the spirit and scope of the present disclosure.

Therefore, the embodiments disclosed herein should be considered in an illustrative aspect rather than a restrictive aspect. The scope of the present disclosure should be defined by the claims rather than the above description, and equivalents to the claims should be interpreted to fall within the present disclosure.

Meanwhile, the methods according to various exemplary embodiments of the present disclosure described above may be implemented as programs and be provided to servers or devices. Therefore, the respective apparatuses may access the servers or the devices in which the programs are stored to download the programs.

In addition, the methods according to various exemplary embodiments of the present disclosure described above may be implemented as programs and be provided in a state in which it is stored in various non-transitory computer-readable media. The non-transitory computer readable medium is not a medium that stores data for a while, such as a register, a cache, a memory, or the like, but means a medium that semi-permanently stores data and is readable by an apparatus. In detail, the various applications or programs described above may be stored and provided in the non-transitory computer readable medium such as a compact disk (CD), a digital versatile disk (DVD), a hard disk, a Blu-ray disk, a universal serial bus (USB), a memory card, a read only memory (ROM), or the like.

Although the embodiments of the disclosure have been illustrated and described hereinabove, the disclosure is not limited to the specific embodiments described above, and may be variously modified by those skilled in the art to which the disclosure pertains without departing from the scope and spirit of the disclosure as claimed in the claims. These modifications should also be understood to fall within the technical spirit and scope of the disclosure.

1. A method of preprocessing bio-component measurement data for growth prediction performed by a computing device, comprising:
receiving physical data of a subject;
generating a first variable based on the physical data of the subject; and
determining an error in the physical data by comparing the first variable with a preset value.

2. The method of claim 1, wherein the receiving of the physical data of the subject includes:
receiving bio-component data of the subject; and
receiving identification data of the subject.

3. The method of claim 2, further comprising:
after receiving the physical data of the subject, generating first data by connecting the bio-component data of the subject and the identification data of the subject.

4. The method of claim 3, further comprising:
after generating the first data, deleting the bio-component data when a preset value is measured in the bio-component data.

5. The method of claim 3, wherein the first variable is generated based on the bio-component data of the subject.

6. The method of claim 5, wherein the first variable is generated by a sum of body fat mass, soft lean mass, and osseous mineral among the bio-component data of the subject.

7. The method of claim 3, wherein the determining of the error includes comparing the first variable with a value of any one of the bio-component data.

8. The method of claim 7, wherein the determining of the error further includes determining whether a monthly age of the subject among the identification data corresponds to a preset criterion before comparing the first variable with a value of any one of the bio-component data.

9. The method of claim 7, further comprising:
when a result value of comparing the first variable with any one value of the bio-component data corresponds to a preset range, deleting the bio-component data.

10. The method of claim 7, further comprising:
when a result value of comparing the first variable with any one value of the bio-component data does not correspond to a preset range, generating second data.

11. A program stored in a computer-readable recording medium including a program code for executing the method of preprocessing bio-component measurement data for growth prediction described in claim 1.

12. A computer-readable recording medium on which a program for executing the method of preprocessing bio-component measurement data for growth prediction described in claim 1 is recorded.

13. An apparatus for preprocessing bio-component measurement data for growth prediction, comprising:
an input unit that receives physical data of a subject;
a variable generation unit that generates a first variable based on the physical data of the subject; and
an error determination unit that determines an error in the physical data by comparing the first variable with a preset value.

14. The apparatus of claim 13, wherein the input unit includes:
a first input unit that receives bio-component data of the subject; and
a second input unit that receives identification data of the subject.

15. The apparatus of claim 14, further comprising:
a connection unit that connects data received through the first input unit and the second input unit.
